(19) United States
(12) Patent Application Publication
(10) Pub. No.: US 2016/0008554 A1
(43) Pub. Date: Jan. 14, 2016

(54) SAFE NEEDLE DEVICE FOR SYRINGES

(71) Applicant: Dietrich Walter Grabis, San Rafael, CA (US)

(72) Inventor: Dietrich Walter Grabis, San Rafael, CA (US)

(21) Appl. No.: 14/853,158

(22) Filed: Sep. 14, 2015

Related U.S. Application Data
(63) Continuation-in-part of application No. 13/723,258, filed on Dec. 21, 2012.

(51) Int. Cl. A61M 5/32

(52) U.S. Cl. CPC A61M 5/3221; A61M 2005/3231; A61M 2005/3224

(57) ABSTRACT

A self-retracting safety syringe has a hollow syringe body, a hollow needle having a point at its tip and a tip-lock at its base, a sleeve around the needle configured to sealingly retain the needle, a hollow plunger configured to engage the interior walls of the syringe body, the plunger having a tip sealingly engaged with the interior walls and having an aperture therein to accommodate the tip-lock, a collar circumferentially mounted on the interior wall of the plunger, a retractor for accepting and retaining the tip-lock, wherein the distal end is retained within the proximal end of the plunger by the collar, and a spring biasing the retractor against the collar, wherein the tip-lock and plunger join, and wherein the retractor is pushed past the collar and is adapted to pull the needle from within the sleeve into the hollow plunger.

### CROSS-REFERENCE TO RELATED APPLICATION

The present application is a continuation in part of U.S. Ser. No. 13/723,258 filed on Dec. 21, 2012 entitled “Safe Needle Device for Syringes” the entire disclosure of which is incorporated by reference herein.

### BACKGROUND OF THE INVENTION

1. Field of Invention

This invention relates to safe hypodermic needles wherein the needle is protected after injection to prevent medical practitioners from receiving needle stick injuries and infections.

2. Description of Related Art

The present invention is directed in general to a safe needle device for syringes as used in the medical industry and hospitals. The “Improvement Patent” (classified as a UTILITY PATENT, according to the USPTO) application presents two significant improvements which are based upon numerous marketability tests (see Commercial Feasibility after the Background section below) conducted over the past several years and after FDA 510(k) approval to market the invention, issued under K010477. The two improvements are:

a) reducing the bulkiness of the device as expressed by Vanderbilt University's Medical School of Nursing, Nashville, Tenn. and other potential users.

b) providing an ergonomically more convenient location and use of the retraction release.

With the advent of AIDS and other highly infectious diseases, syringes and scalpels and other sharps devices have caused numerous injuries and infections to administering medical staff. Protective devices have been introduced to the market after the U.S. legislation in 1998, but consisted of needle covers that still left part of the needle exposed after use and thereby subjecting the clinician to exposure to contaminated blood. These devices are still on the market, but are not user friendly and safe. For example, a nurse must use two hands to prepare a patient for an injection. One hand is used to clean the area to be injected with a disinfectant while the other hand is used to inject the needle into the tissue. After removing the needle from the patient, the administering nurse must press on the spot of injection and hold the syringe in the other hand. After pressing the spot is complete (to stop the flow of blood), the nurse, in prior protective devices, then uses that hand to twist a cover over the syringe or sharp. That requires two hands during which time the needle may drip blood onto the patient or the nurse where contamination is inevitable. In addition, the maneuvering to accomplish the foregoing may cause an inadvertent puncturing of the nurse's own skin, thus inviting infection. One such device is Badger U.S. Pat. No. 5,885,257, which is a very cumbersome releasable retaining device to retract the needle where it will not cause harm. In this Improvement Patent filing the above statements remain unchanged, but the ease of handling the device during administering medication by a clinician is greatly improved and so is increased safety to the patient and clinician. Two mechanically retracting safety syringes came on the market, i.e. the Integra, marketed by BD (Becton Dickenson) of Franklin, N.J., U.S. Pat. No. 5,632,733 (Shaw) and U.S. Pat. No. 7,351,224 (Shaw) and the Vanish Point, marketed by RTI (Retractable Technology, Inc.) of Houston, Tex., and U.S. Pat. No. 6,090,077 (Shaw, 7/2000). While the spring-operated needle retraction is the same in both devices (BD and RTI), they both have their compression springs located in the front end of the syringe whereas the Grabis invention has the spring located inside the plunger. In addition, the Vanish Point does not have a Luer-Lock and, thus, does not permit interchange-ability of needles prior to use. The Integra is hard to push to cause retraction which is a detriment for single hand operation because it can cause significant patient discomfort especially when the device is triggered while the needle is still in the patient. The spring assembly in the Integra (BD) can easily be removed after retraction; it falls apart. In U.S. Pat. No. 5,053,010, McGary states in claim 1. “. . . a cutting tip configured inside said sealing member . . . ” in claim 2 he states “. . . said cutting tip is a beveled knife”. The Grabis invention does not use any cutting tip. It uses a mechanically inter-locking tip-lock. In U.S. Pat. No. 5,211,629, Pressly (10/1991) states “Positioned between sacrificial supports in the needle assembly and the barrel is a deformable base and . . . the plunger has a rupturable boot”. The McGary and Pressly inventions are quite a departure from the Grabis invention. After extensive marketability testing and commercial feasibility evaluation, the Grabis invention was made faster in retraction, smaller in the outer diameter, lighter and cheaper to manufacture.

### SUMMARY OF THE INVENTION

A self-retracting safety syringe has a hollow syringe body, a hollow needle having a point at its tip and a tip-lock at its base, a sleeve around the needle configured to sealingly retain the needle, a hollow plunger configured to engage the interior walls of the syringe body, the plunger having a tip sealingly engaged with the interior walls and having an aperture therein to accommodate the tip-lock, a collar circumferentially mounted on the interior wall of the plunger, a retractor for accepting and retaining the tip-lock, wherein the distal end is retained within the proximal end of the plunger by the collar, and a spring biasing the retractor against the collar, wherein the tip-lock and plunger join, and wherein the retractor is pushed past the collar and is adapted to pull the needle from within the sleeve into the hollow plunger.

In an embodiment, the sleeve has a Luer-lock connection with the proximal end of the body to sealingly retain the needle against the body.

A manually-retracting safety syringe has a hollow syringe body configured to deliver fluids to a patient, a hollow needle having a point at its tip and a tip-lock at its base, wherein the tip-lock comprises a male protrusion configured to engage with end of the plunger, a sleeve around the needle configured to sealingly retain the needle, and a plunger configured to slidingly engage the interior walls of the syringe body, the plunger having distal and proximal ends and a breaking point along its length, the plunger having a tip sealingly engaged with the interior walls of the syringe body, the tip having an aperture therein to accept and retain the tip-lock, wherein when the plunger tip pushes against the tip-lock, the tip-lock is retained by the aperture, and the plunger is adapted to pull the needle from within the sleeve into the syringe body, and wherein the plunger may be broken off at the breaking point to safely retain the needle within the syringe body.

In an embodiment the breaking point is a crease on the plunger, or indents on the plunger.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a cross-sectional view of a syringe mbodying the improved invention showing its basic standard syringe construction with a female Luer-Lock connection and vapor lock.

FIG. 2 is a cross-sectional view of the needle base assembly with needle connection means to allow the needle to disappear inside the plunger after activation.

FIG. 3 shows a cross-sectional view of the retracted needle inside the plunger and the compressed spring in the retention means at the forward end of the plunger.

FIG. 4 shows a side view of the syringe, according to an embodiment of the present invention.

FIG. 5 shows an exploded view of the syringe according to an embodiment of the present invention.

FIG. 6 shows a manual retraction mechanism of the syringe, according to an embodiment of the present invention.

### DETAILED DESCRIPTION OF PREFERRED EMBODIMENTS

Preferred embodiments of the present invention and their advantages may be understood by referring to Figs. (x-y) wherein like reference numerals refer to like elements.

It is a general object of the improvements to the original invention, as shown in patent U.S. Pat. No. 6,322,540 B1 to provide a further improved retractable safety needle device for syringes.

In accordance with the above object, there is provided a needle-stick safety syringe comprising a tubular body, cylindrical plunger, and extended needle at the distal end of the tubular body. Said needle to be removable, through attachment means, in order to be replaced with a different size needle if so desired or required based upon patient needs.

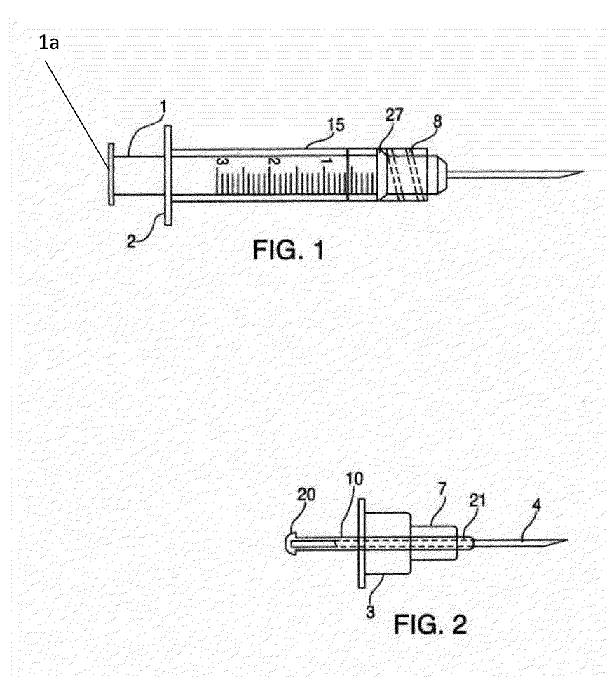

Referring now to FIG. 1, a typical commercial syringe that has a small (for example, 3 ml) capacity and is illustrated with a tubular body 15 and plunger 1 having a connected seal 27 that slides within the tubular body 15 and sealingly and slidingly engages the inside walls of the tubular body 15. The plunger 1 has a flat end 1a adapted to be pushed by the thumb. The tubular syringe body 15 has a flange 2 extending outwardly at its proximal end for engagement with the fingers to permit one-handed operation. The distal end 8 of the tubular body 15 has an extended female Luer-Lock screw-on connector 8 which is an integral part of the syringe body 15. The connector 8 comprises a cylinder projecting from the distal end of the tubular body 15 having internal threading for accepting a needle thereon. Within the cylinder is a tapered nozzle which sealingly engages with the needle when the needle is engaged within the threading. With reference to FIG. 2, the needle base assembly 3 is adapted to be inserted into the connector 8 and be retained there. The needle and sleeve are mounted within the female connector 8.

FIG. 2 shows a needle base assembly 3 providing a receiver to permit extracting the needle 4 from a sleeve 7. The needle 4 is encased in a bonding plastic material within the sleeve at its distal end, as is done with all syringes whether they are safety syringes or standard syringes, and then connected to a retainer 10 providing connecting means to the encased needle 4 through a tip-lock 20 that will engage in the receiver in the plunger 1. The retainer 10 is wedged into the sleeve 7 via a conically shaped collar 21, and a sealant may he used.

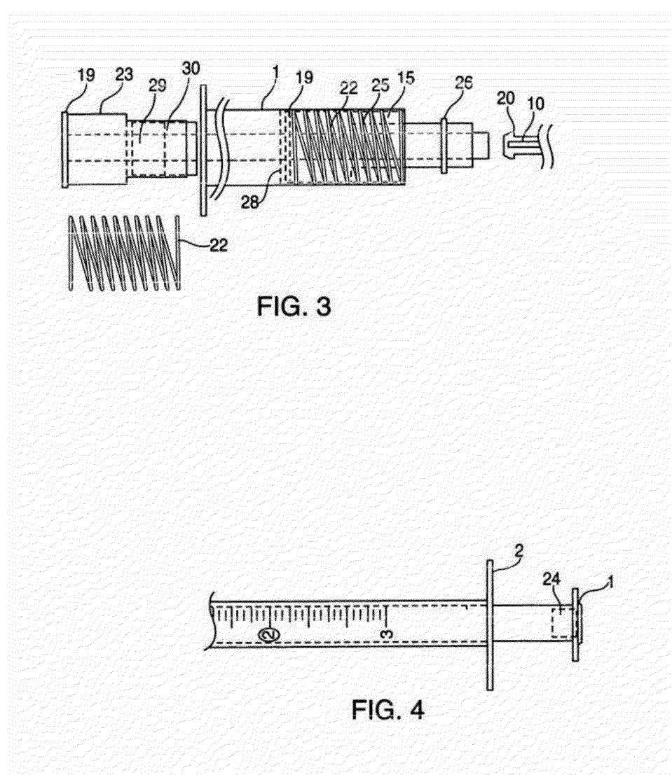

FIG. 3 shows the plunger 1 that houses the compression spring 22. A latch, permitting the retraction of the needle, provide the interconnection between the distal end 26 of the plunger I and the needle base 20, said needle base 20 having a cylindrical body inside a cylindrical adapter. Said adapter having a male thread at its distal end in form of a ring that comprises a single male thread that provides means to engage it into a mating female thread. Said female thread is at the distal end of the syringe body. Said cylindrical body inside said cylindrical adapter performs a dual function. It provides the means to firmly encase the base of the needle 20 and is shaped on the opposite end to provide means to engage the base of the plunger 1.

The coil spring 22 is positioned inside said plunger 1 behind a collar 28 on the interior of the plunger 1 that provides means to interconnect said plunger 1 with said needle base 20. A stop (not shown) inside said plunger is limiting further expansion of said spring 22 after said needle retraction is activated. Through the force of the uncoiling compression spring 22 in the plunger 1, the needle 4 is pulled out of the sleeve 7 and retracted fully into the plunger 1 (see FIG. 3).

The tip-lock engages with the plunger and locks to the plunger tip. The spring serves to retract the needle 4 when activated by the tip-lock, which pushes the receiver through the collar after the needle's tip-lock is connected to the tip of the plunger 1 (see FIG. 2). The spring 22 is compressed between the tip of the cylinder, and a collar 19 within the cylinder so that the spring 22 can not slide over it. The collar 19 serves as a wedge. As the spring 22 uncoils it pushes said cylinder 23 and the locked tip-lock 20 with needle 4 attached to it, to the distal end of the plunger 1. The wedged collar 19 serves two functions. One is to prevent medication from entering the plunger 1 during asperation and injection and the second is to permit the tip-lock 20 (see FIG. 2) to push the collar 19 sufficiently out of its pressure-fitted position so that the spring 22 can uncoil and pull the needle into the plunger 1.

FIG. 4 shows a plug 24, inserted into the end of the plunger 1 by means of a pressure fitting or sonic welding. It prevents the retracted needle from coming out of the back of the plunger 1 after retraction, however the spring also slows expansion naturally as it uncoils.

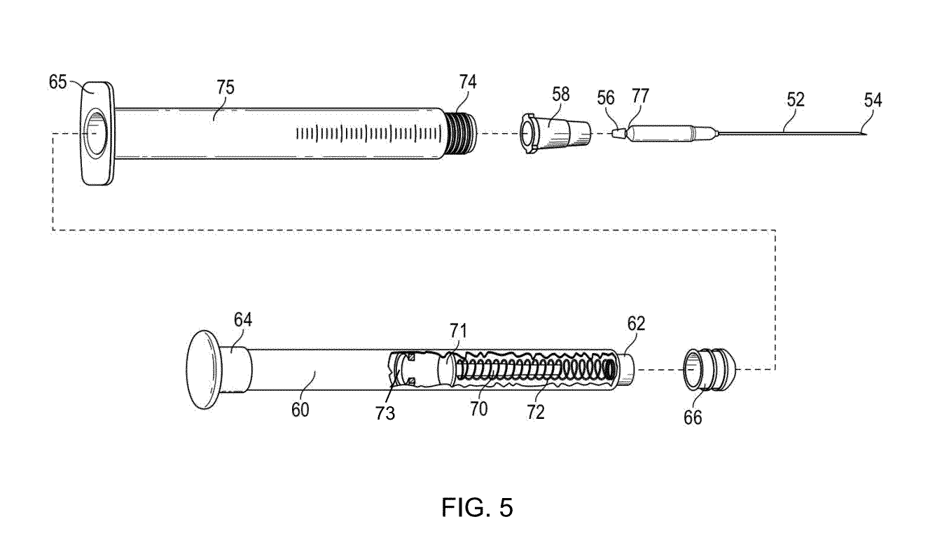

With reference to FIG. 5, in another embodiment the needle extraction mechanism is shown in an exploded view. The needle 52 is hollow for delivering fluids, and has a point 54 at the proximal end and a tip-lock 56 at the distal end. The tip-lock 56 is affixed around the needle 52 and extends therefrom, and is sealingly and frictionally engaged within the male Luer-lock sleeve 58. The sleeve 58 is adapted to engage with the complementary Luer-lock 74 of the syringe cylinder 75. The syringe cylinder is open at the distal end to accept the plunger 60, and it is flared 65 for engagement with the fingers, to depress the plunger. Upon the application of force, the tip-lock 56 and attached needle 52 may be removed from the within the sleeve 58. The needle 52 is seated within the sleeve 58, and in an embodiment, sealed therein. The needle 52 is retained within the sleeve such that it may not proceed through the sleeve 58 in the proximal direction (the direction of the patient), however, it is easily retractable into the distal direction (away from the patient). In an embodiment, the needle 52 is sealed with a sealant.

With further reference to FIG. 5, the plunger 60 is cylindrical and hollow, and contains a collar therein. The plunger 60 is adapted to fit within the syringe cylinder 75. The plunger 60 has a proximal end 62 having a rubber seal 66 that sealingly engages the walls of the cylinder, and has a distal end 64 that is flattened perpendicularly, adapted for engagement with a thumb to push the plunger 60 into the cylinder. The rubber seal 66 has an aperture (not shown) therein providing access to the tip of a retractor 70. The retractor 70 is positioned beneath the collar, wherein the retractor 70 is kept within the proximal end 62 of the plunger 60. A spring 72 is contained between the proximal end 62 and the retractor 70, and biases the retractor 70 against the collar and towards the distal end 64. The retractor 70 has a flare 71 to engage with the spring 72 and prevent the spring 72 from passing over the flare 71. On application of force, the retractor 70 bypasses the collar and is released into the distal, closed end 64 of the cylinder 60, pushed by the spring 72. The collar contains the retractor 70 within the distal end 64 of the plunger 60.

In use, as the plunger 60 descends, the seal 66, attached to the proximal end 62 of the plunger 60, sealingly pressurizes liquids within the cylinder 75 such that they are evacuated out the needle 52, typically into a patient. The needle 52 has a channel therein to permit the passage of fluids therethrough. The tip lock 56, extending from the distal end 64 of the needle, is mounted on supports 77 to permit fluid to flow past the tip-lock 56 into the channel The seal 66 has an aperture therein to permit access to the retractor 70. As the plunger 60 is depressed, the tip-lock 56 enters the retractor 70, and is locked therein. At the proximal end, the retractor 70 has a receiver adapted to receive and retain the tip-lock 56, and a stop to prevent the tip-lock 56 from moving through the retractor 70. As the plunger 60 is further depressed, the tip-lock 56 passes the retractor 70 opening and pushes against the stop, which force causes the distal end of the retractor 70 to push against the collar 73, and move past the collar 73 to enter the distal end of the interior of the plunger 60. As the retractor 70 is spring biased into the distal end of the plunger 60, it moves with some rapidity into the end, pulling the tip-lock 56, and consequently the needle 52, along with it into the body of the plunger 60. The seal 66 aperture is small in relation to the diameter of the plunger 60, and the retracted needle 52 is safely encased therein. The syringe may then be discarded.

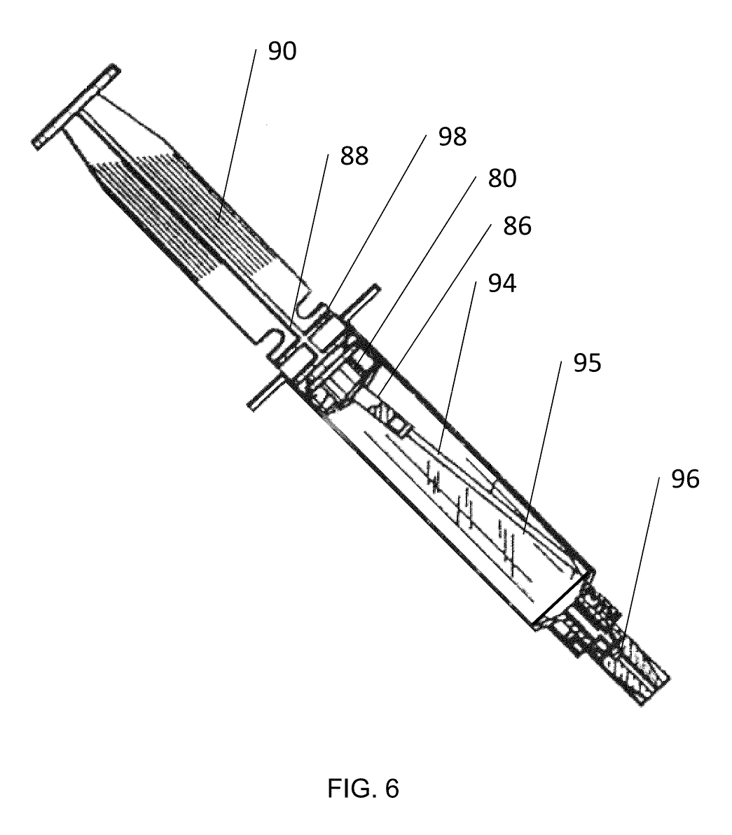

With reference to FIG. 6, a manual embodiment of the retraction mechanism is shown. The plunger seal 80 has a seal aperture (not shown) for engaging with and retaining the tip-lock 86 of the needle, in the same manner as the embodiment above. The plunger 90, which need not be hollow, and has a breaking point 88 such as a crease or indent therein distally of the seal 80, to facilitate the breaking of the plunger 90. Once the seal aperture 82 and tip-lock 86 are engaged, the plunger 90 may be withdrawn, and pull the tip-lock 82 and needle 94 out of its sleeve 96 into the body 95 of the syringe. Once the needle 94 is safely within the body, the plunger 90 is bent along the crease 88 and sheared or broken off, such that the opening of the syringe body is sealed by the plunger and the plunger end 98, and the remaining plunger handle is discarded. After retraction, the needle 94 deflects to one side inside the body 95 to make it impossible to push the needle 94 forward and out of the aperture for re-use.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A self-retracting safety syringe comprising:
a) a hollow syringe body configured to deliver fluids to a patient wherein the body has a proximal end through which the fluids pass and a distal end configured to receive a plunger;
b) a hollow needle having a point at its tip and a tip-lock at its base, wherein the tip-lock comprises a male protrusion configured to engage with end of the plunger;
c) a sleeve around the needle configured to sealingly retain the needle against the proximal end of the body;
d) a hollow plunger configured to slide within the syringe body, the plunger having distal and proximal ends, the plunger comprising:
i) a tip sealingly engaged with the interior walls of the syringe body, the tip having an aperture therein adapted to accommodate the tip-lock;
ii) a collar circumferentially mounted on the interior wall of the plunger;
iii) a refractor having a proximal end in communication with the aperture, configured to accept and retain the tip-lock, and a distal end, retained within the proximal end of the plunger by the collar; and
iv) a spring biasing the retractor against to collar towards the distal end of the plunger;
wherein when the plunger tip pushes against the tip-lock, the tip-lock is retained by the retractor, and wherein the retractor is pushed over the collar wherein the retractor is biased into the distal end of the plunger by the spring and is adapted to pull the needle from within the sleeve into the hollow plunger.

2. The syringe of claim 1 wherein the sleeve has a Luer-lock connection with the proximal end of the body to sealingly retain the needle against the body.

3. A manually-retracting safety syringe, comprising:
a) a hollow syringe body configured to deliver fluids to a patient;
b) a hollow needle having a point at its tip and a tip-lock at its base, wherein the tip-lock comprises a male protrusion configured to engage with end of the plunger;
c) a sleeve around the needle configured to sealingly retain the needle; and
d) a plunger configured to slidingly engage the interior walls of the syringe body, the plunger having distal and proximal ends and a breaking point along its length, the plunger comprising:
i) a tip sealingly engaged with the interior walls of the syringe body, the tip having an aperture therein to accept and retain the tip-lock,
wherein when the plunger tip pushes against the tip-lock, the tip-lock is retained by the aperture, and the plunger is adapted to pull the needle from within the sleeve into the syringe body, and wherein the plunger may be broken off at the breaking point to safely retain the needle within the syringe body.

4. The syringe of claim 2 wherein the breaking point is a crease on the plunger.

5. The syringe of claim 2 wherein the breaking point comprises indents on the plunger.
